(19) United States
(12) Patent Application Publication
(10) Pub. No.: US 2010/0197185 A1
(43) Pub. Date: Aug. 5, 2010

(54) LOW AND ULTRA-LOW FORMALDEHYDE EMISSION BINDERS FOR NON-WOVEN GLASS MAT

(75) Inventor: CHARLES G. HERBERT, Shrewsbury, MA (US)

Correspondence Address: DUANE MORRIS LLP - Philadelphia;IP DEPARTMENT, 30 SOUTH 17TH STREET, PHILADELPHIA, PA, 19103-4196, US

(73) Assignee: Saint-Gobain Technical Fabrics America, Inc.

(21) Appl. No.: 12/362,943

(22) Filed: Jan. 30, 2009

(51) Int. Cl. D04H 13/00; B05D 1/36; C07K 7/06

(52) U.S. Cl. 442/327; 427/203; 530/330

(57) ABSTRACT

Non-woven glass mats and methods of making and using the same are provided. The mats include chopped glass fibers and a curable binder disposed on the glass fibers. The binder contains a modified soy protein and exhibits low to ultra-low or negligible formaldehyde emissions.

### FIELD OF THE INVENTION

This invention concerns a low formaldehyde emitting binder for a non-woven glass mat suitable for use in building construction materials, and methods of making and using the same.

### BACKGROUND OF THE INVENTION

Resin based binders are used to manufacture wet-laid chopped glass fiber mats. Such mats are used in building materials such as roofing shingles, drywall and insulation facers, carpet backing, and air filters. These mats are typically prepared using urea-formaldehyde binders. Urea-formaldehyde based resins emit formaldehyde during curing in glass mat manufacture. The urea-formaldehyde based mat product continues to emit formaldehyde during storage and secondary manufacturing.

In some countries, growing environmental pressures are resulting in current or proposed legislation which may require manufacturers to limit or eliminate formaldehyde emissions from building materials. Accordingly, there is a continued and growing need for compositions suitable for use in building materials that have low or negligible formaldehyde emissions.

The formaldehyde emissions of known urea-formaldehyde resins are at least partly attributable to urea-formaldehyde moisture sensitivity. Moisture sensitivity increases depolymerization hydrolysis reactions in the presence of moisture, i.e. moisture increases the reactions which release formaldehyde. The depolymerization reaction is further accelerated in the presence of acid catalysts that are added to known urea-formaldehyde resins to promote curing of the binder.

It is difficult to avoid exposing a resin to moisture. Because urea-formaldehyde is a hydrophilic polymer system, it has a tendency to adsorb moisture from the air in the surrounding environment. The mat may be exposed to ambient moisture in a glass mat manufacturing plant and/or during transfer of the mat to a secondary manufacturing plant (such as a shingle manufacturing plant). In asphalt shingle manufacturing, for example, a glass mat often picks up moisture during storage after it is manufactured into a rolled mat. Absorbed moisture may then combine with acid residue present in the binder. The resulting combination releases formaldehyde gas when hot asphalt is applied during the shingle manufacturing process.

High temperature asphalt shingle manufacturing occurs generally in the 200-250° C. temperature range as the molten asphalt is applied to the non-woven mat. During this process formaldehyde is evolved from the mat yielding plant emissions that are captured by the ventilation system. The mat is continuously fed into the molten asphalt and a continuous evolution of formaldehyde occurs. If the urea-formaldehyde based mat has not been completely cured there can be relatively large volumes of formaldehyde liberated into the plant environment. Sufficient ventilation must be installed to minimize worker exposure.

Some attempts to reduce formaldehyde emissions are known in the art. Depolymerization can be reduced by adding hydrophobic agents to the binder or through use of a fugitive acid catalyst such as ammonium chloride. The formaldehyde to urea ratio can also be lowered by the resin manufacturer to reduce residual formaldehyde in the system. Still other attempts include addition of low volatility free amines to the binder or adding formaldehyde scavengers. Among the formaldehyde scavengers used as additives in urea-formaldehyde-based binders are melamine, starch, soy proteins, and lignin sulphonates.

A number of compositions for non-woven fabrics which do not emit formaldehyde upon cross-linking have been disclosed in the prior art. See, for example, U.S. Pat. Nos. 5,143,582; 6,734,237; 6,884,838; European Pat. No. EP 0405917 and U.S. Pat. Applications 2006/0292952 and 2007/0039703, the disclosures of all of which are hereby incorporated by reference.

Formaldehyde-free binder chemistry, based upon water-dispersed poly(acrylic acid) blended with polyol and an acid catalyst, has been marketed as an environmentally friendly alternative to urea formaldehyde. Known formaldehyde-free binder chemistries have several drawbacks, however. Acrylic/polyol-based non-woven mats tend to yield sufficient dry tensile strength, but often exhibit insufficient hot wet tensile strength, due to moisture sensitivity. The acrylic/polyol chemistry requires a much higher curing temperature in comparison to urea formaldehyde. The difficulty of providing the required higher curing temperature during production can result in an insufficiently cured mat product. The acrylic/polyol binder becomes undesirably water sensitive if it is insufficiently cured during mat production.

An important property for a non-woven glass mat for use in building construction materials is the ability to retain tensile strength after exposure to heat and moisture. Generally the tear strength and tensile strength of non-woven mats made with conventional formaldehyde-free acrylic binders are within acceptable ranges for urea formaldehyde emission minimums and maximums. Such conventional formaldehyde-free mats, however, have insufficient hot wet strength percent retention. The hot wet percent retention is a quantity defined as the tensile strength of the mat after five minutes of exposure to 80° C. water, multiplied by 100% and then divided by the dry tensile strength of the mat. Hot wet strength percent retention is important for the integrity and durability of the building materials constructed from the glass mat product.

Commercially available formaldehyde-free alternatives to urea-formaldehyde binders are based upon polyacrylic acid blended polyol, typically a triethanol amine. Such binders are not resistant to moisture and result in wet retention percentages of about 52% or less when used to bind non-woven glass mat hand sheets at 200° C. curing temperatures. Products constructed from these binders, while lower in formaldehyde emissions, are inferior to their urea-formaldehyde counterparts regarding strength and durability.

Accordingly, there remains a need for non-woven mats that have low or negligible formaldehyde emissions, yet retain sufficient hot wet tensile strength, dry tensile strength, and tear strength.

### SUMMARY OF THE INVENTION

The invention relates in part to a non-woven mat containing chopped glass fibers and a low formaldehyde emitting binder. The invention also relates in part to the binder, which contains a modified soy polymer or other waterborne soy protein. The invention also relates in part to methods of manufacturing and using the non-woven glass mat and binder.

The steps of a preferred method of this invention include applying chopped glass fibers to a surface, applying the waterborne soy protein-containing binder to the chopped glass fibers, cross-linking the binder, and curing the chopped glass fibers and waterborne soy protein-containing binder to form a mat. The binder can be cross-linked in different ways, depending on the contents of the binder.

In one embodiment, the binder contains urea-formaldehyde. In this embodiment, the steps of the claimed method include applying chopped glass fibers to a surface, adding a modified soy polymer to a urea formaldehyde binder, applying the modified soy polymer and urea formaldehyde binder to the chopped glass fibers, and curing the chopped glass fibers, the modified soy polymer, and the urea formaldehyde binder to form a mat.

In a different embodiment, the binder is cross-linked by an external cross-linking agent. This embodiment of the binder does not require the addition of urea-formaldehyde. Several suitable external cross-linking agents can be used in the claimed method. For example, the external cross-linking agent can contain a reagent with cross-link polyol functionality. Such a reagent may be a tris(alkyoxycarbonylamino)triazine (“TACT triazine”), an epoxy silane, a zirconium ammonium carbonate, a glyoxal, a water dispersible blocked isocyanate, a water dispersible epoxy, or a water dispersible isocyanate. Alternatively, the external cross-linking agent can cure organic acid functionality. Such an agent can be a carbodiimide, an aziridine, a water epoxy, an epoxy silane, or a water dispersed oxazoline. In yet another embodiment, the cross-linking agent used is a cross-linking polymer, such as polyamidoamide epichlorohydrin. In this embodiment, the steps of the method for manufacturing the mat include applying chopped glass fibers to a surface, cross-linking a soy protein-containing binder with a cross-linking polymer, applying the soy protein-containing binder to the chopped glass fibers, and curing the chopped glass fibers and soy protein-containing binder into a mat.

The mat is formulated to emit less than about 40 micrograms per square meter of mat per hour per gram of binder at 23° C. and 50% relative humidity, at about 24 hours after manufacture. The mat is also formulated to emit less than 50% of the emissions by weight formaldehyde during 10 minutes of curing at 180° C., relative to an unmodified urea formaldehyde based mat. The mat emits less than 50% of formaldehyde per gram of binder at 250° C. for five minutes relative to an unmodified urea formaldehyde based control.

The present invention also relates in part to a binder composition for use in manufacturing building construction materials. In one embodiment, the composition contains a urea formaldehyde binder and at least about 10% by dry weight modified soy polymer. In an alternative embodiment, the binder composition contains a modified soy polymer and an external cross-linking agent. The formaldehyde emissions of the binder are less than about 40 micrograms per square meter of mat per hour per gram of binder at 23° C. and 50% relative humidity, at about 24 hours after manufacture.

The cross-linking agent used in one embodiment of the claimed binder can be a cross-linking polymer, such as a polyamidoamide epichlorohydrin. In a different embodiment, the cross-linking agent is a reagent with cross-link polyol functionality. Such a cross-linking agent can be a TACT triazine, an epoxy silane, a zirconium ammonium carbonate, a glyoxal, a water disbursed blocked isocyanate, a water dispersible epoxy, or a water dispensable isocyanate. Alternatively, the cross-linking agent can be one that cures organic acid functionality, such as a carboiimide, an aziridine, a water dispersible epoxy, an epoxy silane, or a water dispersed oxazoline.

The invention also relates in part to a method of using a non-woven glass mat. According to the method, the mat can be incorporated into one a roofing shingle, a drywall facer, a duct board liner, an insulation facer, a carpet backing, or an air filter.

### BRIEF DESCRIPTION OF THE DRAWINGS

The accompanying drawings illustrate preferred embodiments of the invention as well as other information pertinent to the disclosure, in which:

FIG. 1 is a non-woven glass mat 10 showing randomly-oriented chopped glass fibers 12.

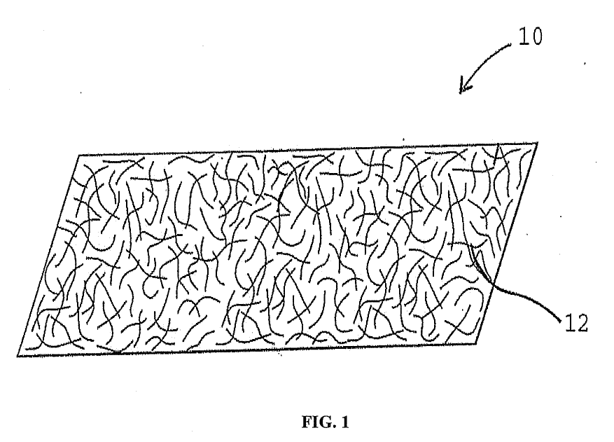

FIG. 2 is a bar graphical depiction of dry and hot wet retention values for soy containing low formaldehyde binders. To obtain the dry tensile strength value, mat hand sheet samples were cut into three 3″×9″ pieces and measured on a tensile testing machine, with 3″ wide grips set apart 7 1/64″, at 2″/min cross head speed. Average values were recorded as pounds per 3″ width. To obtain the hot wet tensile strength value, hand sheets were cut in the same manner as for the dry tensile test and immersed in a controlled temperature water bath set 80° C. for 10 minutes. The samples were quickly blotted to remove excess liquid and tensile tested within 3 minutes by the procedure described above. The percent wet retention is recorded as the hot wet tensile strength divided by the dry tensile strength, and multiplied by 100%.

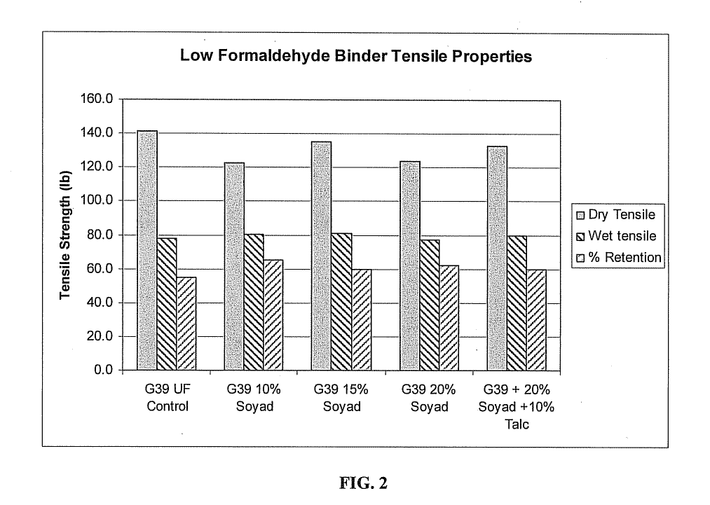

FIG. 3 is a bar graphical depiction of 24 hour emissions for several examples of formaldehyde scavengers compared to a control G39 urea-formaldehyde binder, as described in Example 6.

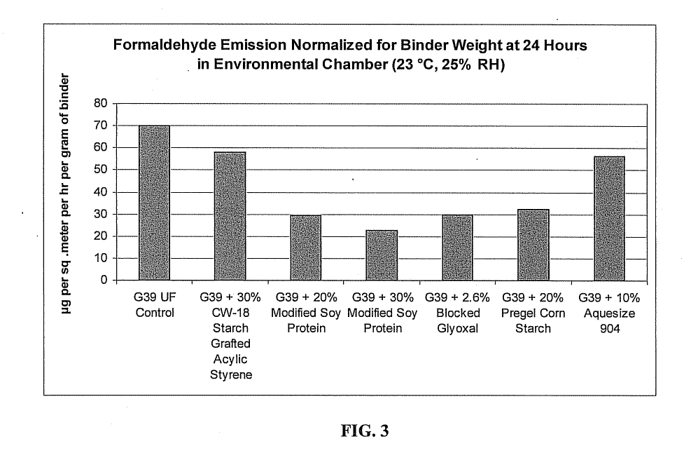

FIG. 4 is a bar graphical depiction of the relative formaldehyde emissions of a soy protein binder and a urea-formaldehyde binder during the simulated mat cure conditions described in Example 7.

FIG. 5 is a bar graphical depiction of the formaldehyde emissions of several soy protein binders and a urea-formaldehyde binder during the simulated roofing shingle production conditions described in Example 8.

FIGS. 6(a) and (b) are line graphical depictions of the relative flexibility of aged and un-aged shingle-grade oxidized asphalt-coated glass hand sheet mats prepared according to the process described in Example 2, and tested for flexibility according to ASTM D3462.

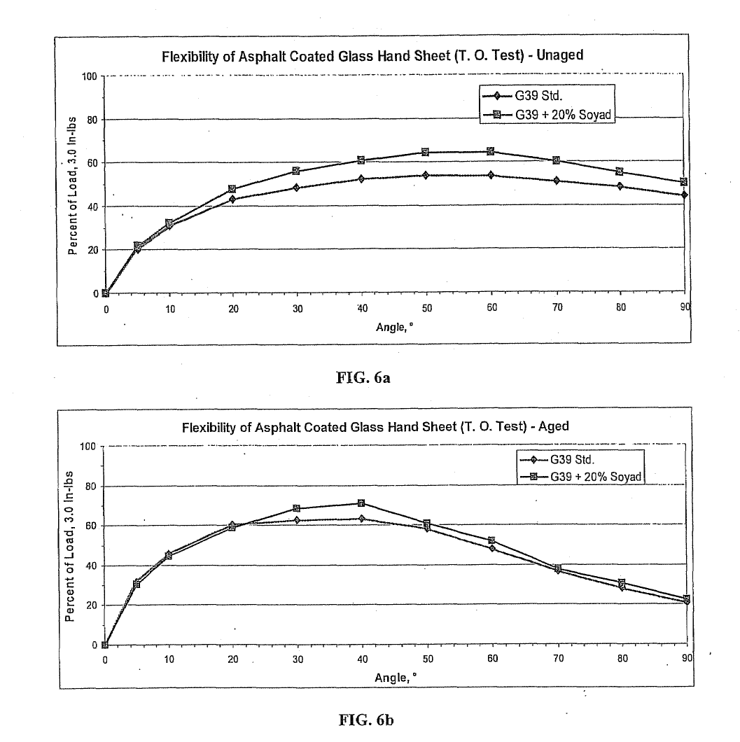

FIG. 7 is a bar graphical depiction of the relative tear strengths of aged and un-aged shingle-grade oxidized asphalt-coated glass hand sheet mats prepared according to the process described in Example 2, and tested for tear strength according to ASTM D3462.

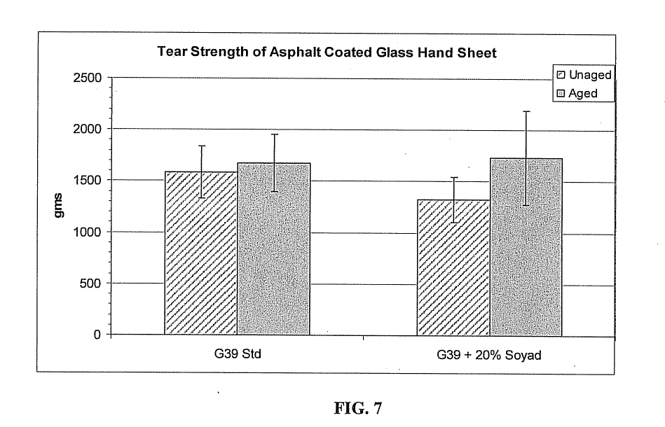

### DETAILED DESCRIPTION OF THE INVENTION

The present invention relates in part to non-woven glass mats or fabrics for use in building construction applications, and methods of making and using these mats. The glass mats include chopped glass fibers, and a curable binder disposed on the glass fibers.

The glass fibers used to form the non-woven glass mats of the present invention may be any type of glass fiber, such as A-type glass fibers, C-type glass fibers, E-type glass fibers, S-type glass fibers, E-CR-type glass fibers, wool glass fibers, or combinations thereof. Wet use chopped strand glass fibers may also be used and can have a moisture content of, for example, about 5-30 wt-%.

The glass fibers may be formed from conventional methods known to those of ordinary skill in the art, for example, the glass fibers may be formed by attenuating streams of molten glass material from a bushing or orifice. The attenuated glass fibers may have diameters of about 5-30 microns, preferably about 10-20 microns. After the glass fibers are drawn from the bushing, an aqueous sizing composition is applied to the fibers. The sizing may be applied by conventional methods such as by an application roller or by spraying the size directly on to the fibers. The size protects the glass fibers from breaking during subsequent processing, helps to retard inter-filament abrasion, and insures an integrity of the strands of glass.

The present invention also relates in part to a curable binder disposed on the glass fibers, which emits low to negligible formaldehyde emissions. The curable binder uses soy protein as a replacement for petroleum derivative products used in conventional urea-formaldehyde binders. The curable binder uses a modified soy protein in a water dispersible liquid form, thereby avoiding the high viscosity associated with soy protein flour additives.

The soy protein based binders and mats disclosed herein exhibit low to ultra-low formaldehyde emissions. Low formaldehyde emissions can be defined as emissions less than about 40 micrograms per square meter of mat per hour per gram of binder at 23° C. and 50% relative humidity, at 24 hours after manufacture. Low formaldehyde emissions can also be defined as emissions of less than 15 micrograms of formaldehyde per gram of binder at 250° C. for five minutes. Ultra-low formaldehyde emissions can be defined as emissions less than about 30 micrograms per square meter of mat per hour per gram of binder at 23° C. and 50% relative humidity, at 24 hours after manufacture, or less than about 5 micrograms of formaldehyde per gram of binder at 250° C. for five minutes.

The non-woven glass mat products disclosed are suitable for use in many industrial, construction, and building material applications. Such applications include, for example, roofing shingle reinforcement, drywall facer, duct board liner, insulation facer, carpet backing, and air filters. The binders disclosed herein emit less formaldehyde than the urea-formaldehyde based binders of the prior art commonly used in these applications.

The binders disclosed herein are based upon modified soy polymers, and more specifically, waterborne modified soy polymers. Two examples of commercially available waterborne modified soy polymers that are suitable for use in the binders and mats disclosed are those sold under the brand names SOYAD and HERCULES.

In one embodiment, soy polymers are added to a urea-formaldehyde binder to reduce formaldehyde emissions during the curing of the binder. In this embodiment, soy protein can be formulated with urea-formaldehyde-based resin alone or in tandem with other formaldehyde scavenging materials. Such materials include, but are not limited to, melamine derivatives, starch, dextrins, casein protein, polyacrylamides, polyaspartic acid, soy protein flour, and starch grafted styrene (for example that sold under the brand name CW-18, SOLV, INC). The addition of soy polymers to urea-formaldehyde binder also reduces formaldehyde emissions of the resulting mat, during transport, storage, and use in further applications.

In addition to reduced formaldehyde emission, the use of modified soy protein provides the advantage of low viscosity for curtain coating on the mat line. There is also an added benefit from the cost standpoint to using a low cost raw material that is based upon renewable natural source not tied directly to natural gas or petroleum feed stocks. The formaldehyde is sequestered via a cross-linking reaction that should make the reverse reaction less favorable at high temperatures.

An example of a curing chemical reaction to scavenge free formaldehyde emitting from a urea-formaldehyde binder containing soy polymers is depicted below:

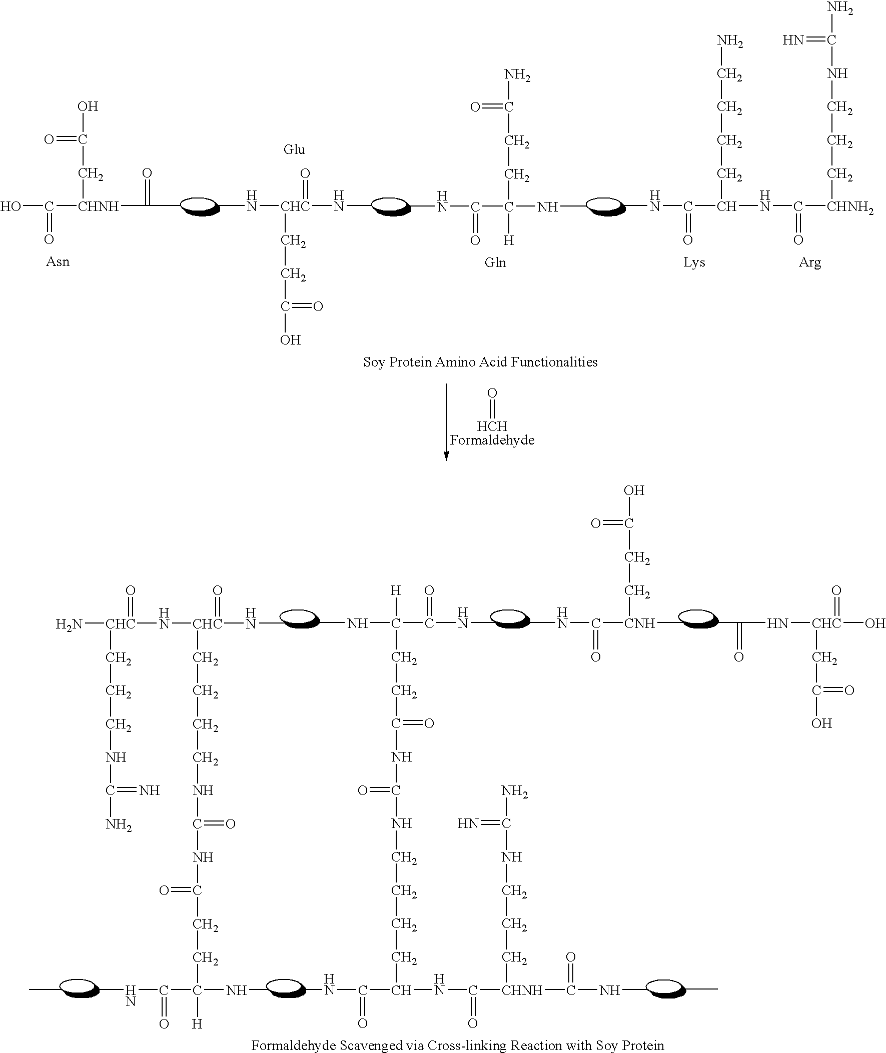

In another embodiment, a soy protein based binder is formed by cross-linking the soy protein in the binder with as cross-linking polymer. In this embodiment, it is not necessary to add a formaldehyde ingredient as part of the binder. Cross-linking polymers suitable for use in the binder of this embodiment include, for example, polyamidoamide epichlorohydrin, or PAAE. Such polymers are commercially available, and examples of suitable polymers are those sold under the brand names KYMENE and HERCULES.

An example of a curing chemical reaction for a soy protein based binder using a cross-linking polymer (PAAE) is depicted below:

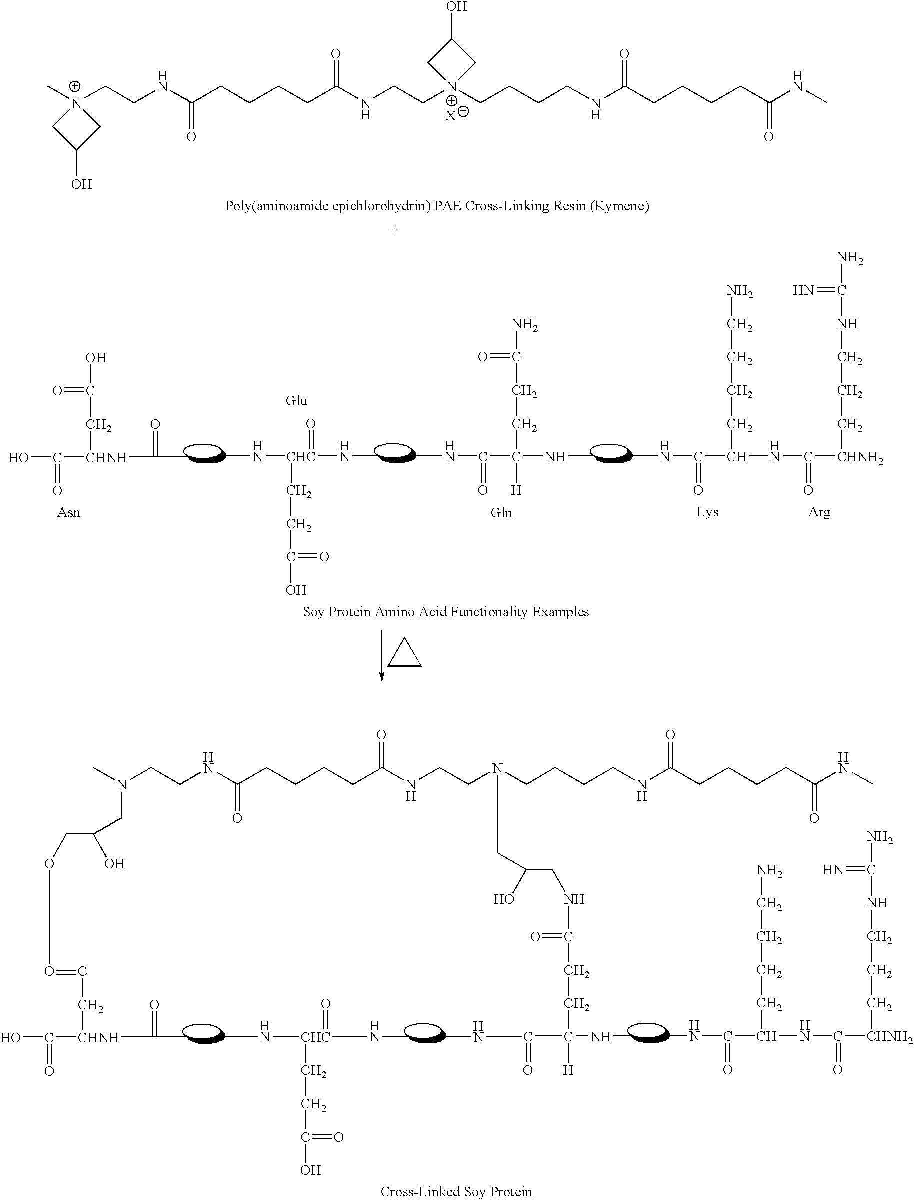

In a further embodiment, the soy protein-based polymer used in the binder disclosed is cross-linked by way of an external cross-linking agent. The external cross-linking agent can be a reagent that cross-links polyol functionality. Examples of suitable reagents for cross-linking a polyol or polyamino functionality include TACT triazine, epoxy silanes, zirconium ammonium carbonate, glyoxal, water dispersed blocked isocyanates, water dispersible epoxies, and water dispensable isocyanates. Suitable formulations of each of these reagents are commercially available. Examples of a suitable TACT triazine are those sold under the brand names CYLINK 2000 and CYTEC. An example of a suitable epoxy silane is sold under the brand name COAT-O 1770 by GE SILICONES. An example of a suitable zirconium ammonium carbonate is sold under the brand name EKA AZC 5880LN. An example of a suitable glyoxal is sold under the brand name EKA RC5550. An example of a suitable water dispersed blocked isocyanate is sold under the brand name API-BI792 by ADVANCED POLYMER INC. An example of a suitable water dispersible epoxy is sold under the brand name API-EC11 by ADVANCED POLYMER INC. An example of a suitable water dispersible isocyanate is sold under the brand name DESMODUR DA-L by BAYER.

The external cross-linking agent can also be a reagent that cures organic acid functionality. Suitable reagents for curing organic acid functionality include, for example, carbodiimides, aziridines, water dispersible epoxies and epoxy silanes, and water dispersed oxazoline. Suitable formulations of each of these reagents are commercially available. An example of a suitable carbodiimide is sold under the brand name XR5580 by STAHL. An example of a suitable aziridine is sold under the brand names XAMA 7 by NOVEON. Examples of suitable water dispersible epoxies and epoxy silanes and water dispersed oxazolines are those sold under the brand names APR-500 by ADVANCED POLYMER INC.

The following examples are provided as an explanatory aid to illustrate specific embodiments of the invention.

### EXAMPLE 1

### Preparation of White Water Slurry and Non-Woven Glass Mat Hand Sheet

A 30 gallon mixing tank fitted with a mechanical stirrer was filled with 110 L of 100° F. water. The stirrer was set to 1800 rpm and 4.70 g of polyacrylamide thickener (for example, those produced under the brand names OPTIMER 9901 or NALCO) was added and allowed to completely disperse for 1-1.5 hrs. To the thickened solution 94.1 g of SHERCOPOL DS 140 ethoxylated alkyl amine anionic surfactant (by LUBRIZOL) was added with stirring and allowed to completely disperse for 1 hour. To this solution, 55 g of mineral oil based defoamer (for example, those produced under the brand names FOAMTROL AF300 and GE BETZ) was added with stirring. Nine liters of the resulting white water solution was then pumped to a 10 gallon stainless steel mixing tank with 4 internal flanges and conical bottom fitted with a mechanical stirrer equipped with a stainless steel impeller designed for fiber dispersion. The stirrer was set to 1800 rpm and 7.64 g of 1⅜″ chopped glass M fiber (produced by OWENS CORNING) was added and dispersed for 5 minutes. A ball valve at the bottom of the tank was then opened and the slurry was poured into a 12″×12″ stainless steel Williams Sheet mold with 1 inch of standing water on the bottom over a removable porous nylon mat. The valve on the sheet mold was then opened and the slurry allowed to drain. The nylon mat covered with the wet fiber mat was then removed from the sheet mold and added the excess white water was removed via a vacuum table fitted with a vacuum slit over which the mat was pulled via a motor and chain.

### EXAMPLE 2

### Preparation of “Low” Formaldehyde Binder Containing Soy

A 20% solids binder solution prepared by adding 266.74 g of G39 (a modified urea formaldehyde binder by GEORGIA PACIFIC) to 644.44 g of white water solution (prepared according to Example 1), and 88.82 g of SOYAD 12UT (a soy dispersion by HERCULES) with mechanical stirring. This solution was evenly applied to the chopped fiber mat (as described in Example 1). The excess was removed using a vacuum table. The uncured mat was placed on a stainless steal wire mesh frame and cured via forced air from the top direction using a Mini-Dryer R-3 textile oven manufactured by GATE VADUZ AG. The sample was cured for 3 minutes at 180° C. for 3 minutes. The target basis weight was 1.8 lb/100 sq. ft.

### EXAMPLE 3

### Alternative Formulation for “Low” Formaldehyde Binder Containing Soy

A 20% solids binder solution was prepared by adding 300.03 g of G39 to 655.57 g of white water solution and 44.40 g of SOYAD 12UT with mechanical stirring. This solution was evenly applied (as described in Example 2) to the chopped fiber mat (as described in Example 1). The excess was removed using a vacuum table. The uncured mat was placed on a stainless steal wire mesh frame and cured via forced air from the top direction using a Mini-Dryer R-3 textile oven manufactured by GATE VADUZ AG. The sample was cured for 3 minutes at 180° C. for 3 minutes.

### EXAMPLE 4

### Preparation of “Ultra-Low” Formaldehyde Binder Using Soy

A 20% solids binder solution was prepared by adding of 111.11 g of Soyad 12GT (Soy dispersion, Hercules, Inc) to 138.89 g of white water solution followed by 50 g of Kymene CA1000 cross-linker (polyamidoamide epichlorohydrin, Hercules) This solution was evenly applied (as described in Example 2) to the chopped fiber mat (as described in Example 1). The excess was removed using a vacuum table. The uncured mat was placed on a stainless steal wire mesh frame and cured via forced air from the top direction using a Mini-Dryer R-3 textile oven manufactured by Gate Vaduz AG. The sample was cured for 3 minutes at 180° C. for 3 minutes.

### EXAMPLE 5

### Alternative Formulation of “Ultra-Low” Formaldehyde Binder Using Soy

A 20% solids binder solution was prepared by adding of 95.24 g of Soyad 12GT to 119.05 g of white water solution followed by 85.71 g of Kymene CA1000 cross-linker. This solution was evenly applied (as described in Example 2) to the chopped fiber mat (as described in Example 1). The excess was removed using a vacuum table. The uncured mat was placed on a stainless steal wire mesh frame and cured via forced air from the top direction using a Mini-Dryer R-3 textile oven manufactured by Gate Vaduz AG. The sample was cured for 3 minutes at 180° C. for 3 minutes.

### EXAMPLE 6

### Testing of 24 Hour Emissions for Several Examples of Formaldehyde Scavengers

Glass mat hand sheets were prepared using 1¼″ M fiber targeting shingle mat weight and binder content, 1.86 lb and 20% LOI, respectively. All of the mats, including the control, were cured at 180° C. and sealed in polymer lined aluminum envelops before being tested in the environmental chamber. Formaldehyde emissions were collected using an environmental chamber set at 23° C., 50% relative humidity conditions, and other parameters specified in the “GreenGuard Procedure”. (See Air Quality Sciences Inc; GGTM.PO66.R2-070702 Method for Measuring Chemical Emissions From Various Sources Using Dynamic Environmental Chambers, GreenGuard Publications, Inc., 2006). The soy containing binder exhibited significant formaldehyde reduction over and above the dilution level.

### EXAMPLE 7

### Simulated Mat Cure Conditions

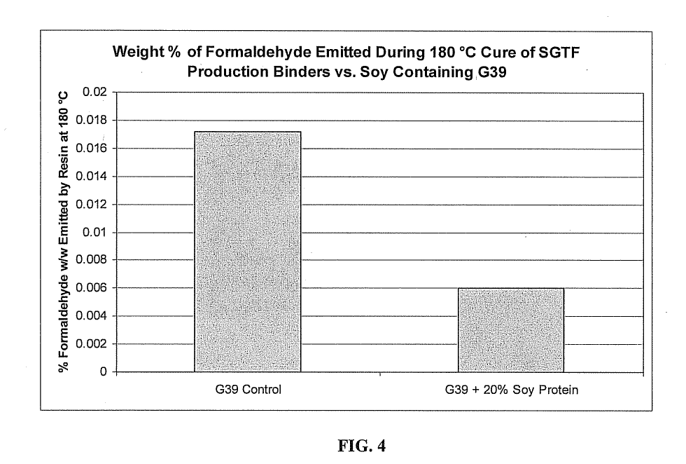

A hand sheet textile oven fitted with a vacuum and water trap. The textile oven was designed to approximate the cure of non-woven mat on a production line. The formaldehyde was captured with a water trap under a constant vacuum applied to the sole exhaust outlet. Upon preparation of a 2,4-dinitrophenylhydrazine derivative, the emission was evaluated via high performance liquid chromatography (HPLC) for various binder chemistries. Using this approach, the binders were cured as dilute (12% solids in aluminum pans) wet solutions for 3 minutes to yield thin films at 180° C. The formaldehyde was captured for 10 minutes, including the cure dwell time. Blank samples were collected after each experimental sample to ensure that the formaldehyde remaining in the oven was restored to baseline values. The relative formaldehyde emission during simulated mat cure conditions was evaluated for the low formaldehyde binders versus the G39 control. The results of this evaluation are depicted in FIG. 4.

### EXAMPLE 8

### Simulated Roofing Shingle Manufacture Conditions

High temperature roofing shingle manufacturing occurs generally in the 200-250° C. temperature range as molten asphalt is applied to the non-woven mat. During this process, formaldehyde is emitted from the mat. These emissions are captured by the ventilation system of the shingle manufacturing plant. The production line generally moves at 400-600 feet/min, so the exposure period is not excessively long. As the mat is continuously fed into the molten asphalt a continuous evolution of formaldehyde occurs. If the urea-formaldehyde based mat has not completely cured, then there can be relatively large volumes of formaldehyde liberated into the plant environment. Sufficient ventilation must be installed to minimize worker exposure.

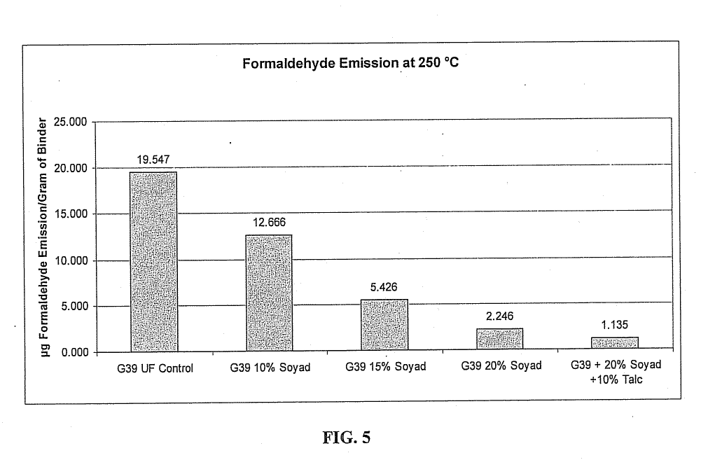

Shingle manufacturing conditions were simulated in a laboratory to evaluate various binder chemistries in terms of their formaldehyde emissions. Under these simulated conditions, glass mat hand sheets were fed into a textile oven for 30 seconds at 250° C. in tandem with 5 minutes capture of emissions using a vacuum water trap under constant vacuum. The results of this simulated secondary manufacturing formaldehyde emission evaluation of low formaldehyde chemistries are shown in FIG. 5.

### Glass Mat Production Examples

On a non-woven mat production line equipped with a honeycomb style forced air oven, the G39 UF resin combined with 15% and 20% soy binder (dry weight on dry weight) formulations were used to prepare 1.8 lb/100 sq. ft. basis weight mat an exit web temperature of 180-190° C. The resulting mat had properties and loss on ignition values are listed below in Table 1, including data for glass mat hand sheets prepared according to the processes described in Examples 2, 3, 4, and 5.

| | Basis | MD | CD | MD | CD | | | |
|---|---|---|---|---|---|---|---|---|
| Production Example | Wt | Tensile | Tensile | Tear | Tear | LOI | Retention | Caliper |
| UF + 20% Soy per | 1.79 | 97.4 | 51.8 | 664 | 882 | 20.57 | 60.3 | 32.44 |
| Example 2 | | | | | | | | |
| UF + 20% Soy per | 1.77 | 78 | 45.3 | 771 | 915 | 19.47 | 55 | 32.53 |
| Example 3 | | | | | | | | |
| UF + 20% Soy per | 1.79 | 71.8 | 45 | 785 | 848 | 17.13 | 66 | 32.34 |
| Example 4 | | | | | | | | |
| UF + 15% Soy per | 1.79 | 83.2 | 50.4 | 834 | 1155 | 18.6 | 63.3 | 32.21 |
| Example 2 | | | | | | | | |
| UF + 15% Soy per | 1.8 | 80.9 | 42.9 | 680 | 973 | 19.83 | 74 | 32.82 |
| Example 3 | | | | | | | | |
| UF + 15% Soy per | 1.79 | 91.3 | 40.8 | 563 | 934 | 19.2 | 65 | 33.07 |
| Example 4 | | | | | | | | |
| UF + 15% Soy per | 1.8 | 87.8 | 43.5 | 662 | 1070 | 18.13 | 70 | 34.3 |
| Example 5 | | | | | | | | |
| G39 UF Control per | 1.8 | 97.6 | 48.2 | 541 | 810 | 21.5 | 72 | 32.13 |
| Example 2 | | | | | | | | |
| G39 UF Control per | 1.78 | 97.4 | 55.4 | 633 | 835 | 20.33 | 78 | 35.24 |
| Example 3 | | | | | | | | |
| G39 UF Control per | 1.79 | 100.2 | 57.5 | 596 | 860 | 19.77 | 69 | 34.43 |
| Example 4 | | | | | | | | |
| G39 UF Control per | 1.79 | 91.7 | 40 | 529 | 859 | 20.47 | 82 | 34.15 |
| Example 5 | | | | | | | | |

UF = Urea-Formaldehyde
MD = Machine direction
CD = Cross-machine direction
LOI = Loss On Ignition
Tensile strength is in lb.
LOI is in percent.
Basis wt. is lb/100 sq. ft.
Tears is in grams.
Caliper is in mils.

What is claimed:

1. A non-woven mat comprising:
(a) chopped glass fibers; and
(b) a binder containing at least about 10% by dry weight of a waterborne soy protein.

2. The mat of claim 1 wherein said waterborne soy protein binder comprises a modified soy polymer.

3. A method of using the mat of claim 1, wherein the method comprises incorporating the mat into one of a roofing shingle, a drywall facer, a duct board liner, an insulation facer, a carpet backing, built up roofing, and an air filter.

4. A method of manufacturing a non-woven mat, the method comprising:
(a) applying said chopped glass fibers to a surface;
(b) cross-linking said waterborne soy protein-containing binder with an external cross-linking agent;
(c) applying said waterborne soy protein-containing binder to said chopped glass fibers; and
(d) curing said chopped glass fibers and said waterborne soy protein-containing binder to form a mat.

5. The method of claim 4 wherein said external cross-linking agent comprises a reagent that will cross-link polyol or polyamine functionality.

6. The method of claim 5 wherein said external cross-linking agent is one of a TACT triazine, an epoxy silane, a zirconium ammonium carbonate, a glyoxal, a water dispersed blocked isocyanate, a water dispersible epoxy, and a water dispensable isocyanate.

7. The method of claim 4 wherein said external cross-linking agent comprises an agent that cures organic acid functionality.

8. The method of claim 7 wherein said external cross-linking agent is one of a carbodiimide, an aziridine, a water dispersible epoxy, an epoxy silane, and a water dispersed oxazoline.

9. A method of manufacturing a non-woven mat, the method comprising:
(a) applying chopped glass fibers to a surface;
(b) adding a modified soy polymer to a urea formaldehyde binder;
(c) applying said modified soy polymer and urea formaldehyde binder to said chopped glass fibers; and
(d) curing said chopped glass fibers, said modified soy polymer, and said urea formaldehyde binder to form a mat, wherein the mat exhibits formaldehyde emissions less than about 40 micrograms per square meter of mat per hour per gram of binder at 23° C. and 50% relative humidity, at about 24 hours after manufacture.

10. A method of manufacturing a non-woven mat, the method comprising:
(a) applying chopped glass fibers to a surface;
(b) cross-linking a soy protein-containing binder with a cross-linking polymer;
(c) applying said soy protein-containing binder to the chopped glass fibers; and
(d) curing said chopped glass fibers and said soy protein-containing binder into a mat.

11. The method of claim 10 wherein the cross-linking polymer is polyamidoamide epichlorohydrin.

12. A binder composition for use in manufacturing building construction materials, the composition comprising:
(a) a urea formaldehyde binder; and
(b) at least about 10% by dry weight modified soy polymer.

13. A method of manufacturing a non-woven mat, the method comprising:
(a) applying randomly-oriented chopped glass fibers to a surface;
(b) applying the binder composition of claim 12 to the chopped glass fibers; and
(c) curing the chopped glass fibers and the composition to form a non-woven mat.

14. A binder composition for use in manufacturing building construction materials, the composition comprising:
(a) a modified soy polymer; and
(b) a cross-linking agent, wherein a formaldehyde emission of the composition is less than 50% relative to an unmodified urea formaldehyde based mat of the same construction per square meter of mat per hour per gram of binder at 23° C. and 50% relative humidity, at about 24 hours after manufacture.

15. The composition of claim 14, wherein said cross-linking agent is a cross-linking polymer.

16. The composition of claim 15, wherein said cross-linking polymer is polyamidoamide epichlorohydrin.

17. The composition of claim 14, wherein said cross-linking agent is a reagent reactive with polyol, polyamine, and polyamide functionality.

18. The composition of claim 17, wherein said cross-linking agent is one of a TACT triazine, an epoxy silane, a zirconium ammonium carbonate, a glyoxal, a water dispersed blocked isocyanate, a water dispersible epoxy, and a water dispersible isocyanate.

19. The composition of claim 14, wherein the cross-linking agent comprises an agent that cures organic acid functionality.

20. The composition of claim 19, wherein the cross-linker is one of carbodiimide, an aziridine, a water dispersible epoxy, an epoxy silane, and a water dispersible oxazoline.

21. A method of making a mat, the method comprising:
(a) applying chopped glass fibers to a surface;
(b) applying the binder composition of claim 14 to the chopped glass fibers; and
(c) curing the chopped glass fibers and the composition to form a non-woven mat.

22. A method of manufacturing a non-woven mat, the method comprising:
(a) applying chopped glass fibers to a surface;
(b) adding a modified soy polymer to a urea formaldehyde binder;
(c) applying said modified soy polymer and urea formaldehyde binder to said chopped glass fibers; and
(d) curing said chopped glass fibers, said modified soy polymer, and said urea formaldehyde binder to form a mat, wherein the mat exhibits a formaldehyde emission which is less than 30% of formaldehyde per gram of binder at 250° C. for five minutes relative to an unmodified urea formaldehyde based mat of the same construction.

23. A method of manufacturing a non-woven mat, the method comprising:
(a) applying chopped glass fibers to a surface;
(b) adding a modified soy polymer to a urea formaldehyde binder;
(c) applying said modified soy polymer and urea formaldehyde binder to said chopped glass fibers; and
(d) curing said chopped glass fibers, said modified soy polymer, and said urea formaldehyde binder to form a mat, wherein the mat exhibits a formaldehyde emission which is less than 50% by weight formaldehyde per gram of binder relative to an unmodified urea formaldehyde based mat of the same construction during 10 minutes of curing at 180° C.
